SEQUENCE LISTING (37 sequences)

SEQ ID NO: 1 - protein, 42 aa, Homo sapiens
PSSVIDALQYKLEGTTRLTRKRGLKLATALSLSNKFVEGSPS

SEQ ID NO: 2 - protein, 19 aa, Homo sapiens
VDRVRLASHLRKLRKRLLR

SEQ ID NO: 3 - protein, 59 aa, Homo sapiens
RRPDFCLEPPYTGPCKARIIRYFYNAKAGLCQTFVYGGCRAKRNNFKSAEDCMRTCGGA

SEQ ID NO: 4 - DNA, 43 bp, Artificial Sequence
  synthetic oligonucleotide
catgggtgcgagagcgtcagtattaagcgggggagaattagat

SEQ ID NO: 5 - DNA, 41 bp, Artificial Sequence
  synthetic oligonucleotide
cgatctaattctcccccgcttaatactgacgctctcgcacc

SEQ ID NO: 6 - DNA, 12 bp, Artificial Sequence
  synthetic oligonucleotide
agcttccgcgga

SEQ ID NO: 7 - DNA, 12 bp, Artificial Sequence
  synthetic oligonucleotide
gatctccgcgga

SEQ ID NO: 8 - protein, 27 aa, Homo sapiens
FIPLKPTVKMLERSNHVSRTEVSSNHV

SEQ ID NO: 9 - protein, 26 aa, Bos taurus
FIPLKPTVKMLERSNVSRTEVSNNHV

SEQ ID NO: 10 - protein, 27 aa, Mus musculus
FIPMKRSVKRLQDQPNIQRTEVNTNHV

SEQ ID NO: 11 - protein, 27 aa, Rattus sp.
FIPVKPSVKKLQDQSNIQRTEVNTNHV

SEQ ID NO: 12 - protein, 27 aa, Mus musculus
FIPLKPTVKKLERLEHVSRTEVSNNNV

SEQ ID NO: 13 - protein, 27 aa, Mus musculus
FIPLKPTVKKLERLEHISRTEVSNNNV

SEQ ID NO: 14 - protein, 27 aa, Rattus sp.
FIPLKPTVKKLERLGHVSRTEVTTNNV

SEQ ID NO: 15 - protein, 27 aa, Rattus sp.
FIPLKPTVKMLERSVHVSRTEVSNNHV

SEQ ID NO: 16 - protein, 27 aa, Homo sapiens
FIPLKPTVKMLERSSSVSRTEVSNNHV

SEQ ID NO: 17 - protein, 28 aa, Homo sapiens
FAIQKIRVKAGETQKKVIFCSREKVSHL

SEQ ID NO: 18 - protein, 28 aa, Mus musculus
FVIERIRVKAGETQKKVIFCAREKVSHL

SEQ ID NO: 19 - protein, 28 aa, Rattus sp.
FVIEKIRVKAGETQKKVIFCAREKVSHL

SEQ ID NO: 20 - protein, 28 aa, Ovis aries
FDIGKIRVKAGETQKKVIFCSREKMSYL

SEQ ID NO: 21 - protein, 28 aa, Bos taurus
FDIGKIRVKAGETQKKVIFCSREKMSYL

SEQ ID NO: 22 - protein, 28 aa, Sus scrofa
FAIEKIRVKAGETQKKVIFCSREKKSHL

SEQ ID NO: 23 - protein, 28 aa, Cavia porcellus
FTIEKIRVKAGETQKKIVFCSREKVSKL

SEQ ID NO: 24 - protein, 28 aa, Gallus gallus
FTIQRVRVKSGETQKKVVFCSRDGSSRL

SEQ ID NO: 25 - protein, 28 aa, Mus musculus
LILKTIWVKAGETQQRMTFCPENLDDLQ

SEQ ID NO: 26 - protein, 26 aa, Homo sapiens
FRLFRSTVKQVDFSEVERARFIINDW

SEQ ID NO: 27 - protein, 26 aa, Mus musculus
FKLFQTMVKQVDFSEVERARFIINDW

SEQ ID NO: 28 - protein, 26 aa, Rattus sp.
FKLFRTTVKQVDFSEVERARFIINDW

SEQ ID NO: 29 - protein, 26 aa, Bos taurus
FRLFRTTVKQVDFSEVERARFIVNDW

SEQ ID NO: 30 - protein, 27 aa, Homo sapiens
TKELGYTVKKHLQDLSGRISRARHNEL

SEQ ID NO: 31 - protein, 27 aa, Rattus sp.
TKELGYKVKKHLQDLSSRVSRARHNEL

SEQ ID NO: 32 - protein, 27 aa, Mus musculus
TKELGYKVKKHLQDLSSRVSRARHNEL

SEQ ID NO: 33 - protein, 28 aa, Homo sapiens
LAKYGLDGKKDARQVTSNSLSQTQEDGL

SEQ ID NO: 34 - protein, 28 aa, Rattus sp.
LARYGLDGRKDTQTVHSNALNEDTQDEL

SEQ ID NO: 35 - protein, 28 aa, Mus musculus
LARYGLDGRKDAQMVHSNALNEDTQDEL

SEQ ID NO: 36 - protein, 27 aa, Homo sapiens
WCYVFKAGKYSSEFCSTPACSEGNSDC

SEQ ID NO: 37 - protein, 21 aa, Homo sapiens
CRAKRNNFKSAEDCMRTCGGA

(19) United States
(12) Patent Application Publication
(10) Pub. No.: US 2010/0015117 A1
(43) Pub. Date: Jan. 21, 2010

(54) COMPOSITIONS AND METHODS FOR TARGETING A POLYPEPTIDE TO THE CENTRAL NERVOUS SYSTEM

(75) Inventors: Inder M. Verma, La Jolla, CA (US); Brian Spencer, San Diego, CA (US)

Correspondence Address: KLARQUIST SPARKMAN, LLP, 121 S.W. SALMON STREET, SUITE 1600, PORTLAND, OR, 97204, US

(73) Assignee: The Salk Institute for Biological Studies

(21) Appl. No.: 12/499,641

(22) Filed: Jul. 8, 2009

Related U.S. Application Data
(63) Continuation of application No. 11/401,604, filed on Apr. 10, 2006.
Application No. 11/401,604 is a continuation of application No. 10/861,779, filed on Jun. 4, 2004, now abandoned.
(60) Provisional application No. 60/476,482, filed on Jun. 5, 2003.

(51) Int. Cl. A61K 38/43; A61K 38/16; C07H 21/04; C12N 15/63; A61K 31/7088; A61P 25/00

(52) U.S. Cl. 424/94.1; 514/12; 536/23.1; 435/320.1; 514/44 R

(57) ABSTRACT

The invention provides a chimeric CNS targeting polypeptide having a BBB-receptor binding domain and a payload polypeptide domain. The chimeric CNS targeting polypeptide can have a BBB-receptor binding domain consisting of a receptor binding domain from ApoB, ApoE, aprotinin, lipoprotein lipase, PAI-1, pseudomonas exotoxin A, transferrin, α2-macroglobulin, insulin-like growth factor, insulin, or a functional fragment thereof. Nucleic acids encoding a chimeric CNS targeting polypeptide are also provided. Further provided is a method of delivering a polypeptide to the CNS of an individual. The method consists of administering to the individual an effective amount of a chimeric CNS targeting polypeptide, said chimeric CNS targeting polypeptide comprising a BBB-receptor binding domain and a payload polypeptide domain. The method also can deliver a polypeptide to the lysosomes of CNS cells.

### CROSS-REFERENCE TO RELATED APPLICATIONS

This application is a continuation of U.S. application Ser. No. 11/401,604, filed Apr. 10, 2006, which is a continuation of U.S. application Ser. No. 10/861,779, filed Jun. 4, 2004 (abandoned), which claims priority to and benefit of U.S. Provisional Application No. 60/476,482, filed Jun. 5, 2003, entitled COMPOSITIONS AND METHODS FOR TARGETING A POLYPEPTIDE TO THE CENTRAL NERVOUS SYSTEM, each of which applications is incorporated herein by reference.

### STATEMENT OF GOVERNMENT SUPPORT

This invention was made with government support under grant number HL-53670 awarded by the National Institutes of Health. The United States Government has certain rights in this invention.

### BACKGROUND OF THE INVENTION

This invention relates to biopharmaceuticals in the treatment of diseases and, more specifically, to the production and delivery of a biopharmaceutical for polypeptide replacement therapy.

Inherited lysosomal disorders occur in approximately 1 in 8000 births worldwide resulting from deficient activity of a key enzyme involved in catalysis of glucosaminoglycans. Symptoms of such disorders range from skeletal deformities to progressive neuronal degeneration. For example, Gaucher's disease is caused by a deficiency of the lysosomal enzyme glucocerebrosidase (GC).

Mucopolysaccharidoses (MPS) are a group of ten of such inherited metabolic disorders caused by a deficiency of a lysosomal enzyme involved in the degradation of mucopolysaccharides. The deficiency leads to an accumulation of the metabolic precursors in the lysosomes and dysfunction of the affected cells. Clinical phenotypes vary with the specific enzyme involved but typically include hepatosplenomegaly, degenerative skeletal defects and even decreased life span. Lysosomal storage disorders also include some degree of neuronal cell loss resulting in mental retardation, physical disability, a decreased life span or a combination of these symptoms.

Current therapies include allogenic bone marrow transplant (BMT) and enzyme replacement therapy (ERT). Although bone marrow transplantations have contributed to treatments in cases of MPS I, II and VI, the correction of hematopoietic cells has not progressed to the level needed to predictably treat the enzyme deficiency disorder. For example, successful bone marrow transplantations for the treatment of neurological symptoms has resulted in limited success. In addition, allogenic bone marrow transplants rely on identifying a closely matched donor and further carries the risk of graft versus host disease.

Enzyme replacement therapy has been attempted with Gaucher's disease, Hunter's disease and Fabry Syndrome and has shown sporadic contributions to the treatment of only milder forms of these diseases. Treatment involves the in vitro modification of recombinant forms of the enzyme deficient in these diseases followed by infused into the patient several times a week for the lifetime of the individual. Although enzyme replacement therapy can be successful in the treatment of a peripheral disease, the infused enzyme does not cross the blood-brain barrier (BBB).

The BBB is composed of a tightly packed layer of endothelial cells and numerous glial or astrocytic process that regulate the passage and diffusion of protein and growth factors from the blood stream to the CNS. Transport of almost all particles to the CNS occurs via binding to specific receptors on the vascular side of the endothelial cell followed by endocytosis and transport to the CNS. Therefore, delivery of proteins by vascular distribution to the CNS is not possible due to the presence of this blood-brain barrier. Accordingly, infusion or other type of administration or delivery of a soluble polypeptide has little effect on the neuronal component of the above neuronal diseases or other lysosomal storage diseases or on neuropathothologies.

Thus, there exists a need for a mode or method that allows the passage of a specific therapeutic polypeptide across the blood-brain barrier. The present invention satisfies this need and provides related advantages as well.

### SUMMARY OF THE INVENTION

The invention provides a chimeric CNS targeting polypeptide having a BBB-receptor binding domain and a payload polypeptide domain. The chimeric CNS targeting polypeptide can have a BBB-receptor binding domain consisting of a receptor binding domain from ApoB, ApoE, aprotinin, lipoprotein lipase, PAI-1, pseudomonas exotoxin A, transferrin, α2-macroglobulin, insulin-like growth factor, insulin, or a functional fragment thereof. Nucleic acids encoding a chimeric CNS targeting polypeptide are also provided. Further provided is a method of delivering a polypeptide to the CNS of an individual. The method consists of administering to the individual an effective amount of a chimeric CNS targeting polypeptide, said chimeric CNS targeting polypeptide comprising a BBB-receptor binding domain and a payload polypeptide domain. The method also can deliver a polypeptide to the lysosomes of CNS cells.

### BRIEF DESCRIPTION OF THE DRAWINGS

FIG. 1 shows the amino acid binding sequences for α2-macroglobulin receptor and LDL related receptor (SEQ ID NOs. 8-37 sequentially from top to bottom).

FIG. 2 shows polypeptide staining of HepG2 cell lysates co-cultured with 293T cells transfected with various GC expressing chimeric CNS targeting polypeptide constructs.

FIG. 3 shows a schematic diagram of a nucleic acid encoding a chimeric CNS targeting polypeptide PPTGCmXfT construct inserted into a 3rd generation lentivirus vector under the control of the CAG promoter.

FIG. 4 shows glucocerebrosidase enzyme activity of liver and brain cell homogenates following intravenous injection of lentiviral vectors containing PPTGCmXfT encoding chimeric CNS targeting polypeptides.

FIG. 5 shows liver sections of animals intravenous injection with lentiviral vectors containing PPTGCmXfT encoding chimeric CNS targeting polypeptides that are shown in FIG. 4.

FIG. 6 shows whole brain sections of animals intravenous injection with lentiviral vectors containing PPTGCmXfT encoding chimeric CNS targeting polypeptides that are shown in FIG. 4.

### DETAILED DESCRIPTION OF THE INVENTION

This invention is directed to the identification of modular targeting molecules that can selectively penetrate the blood-brain barrier (BBB). The targeting molecules can carry and deliver any polypeptide of interest to the central nervous system (CNS). Such CNS targeting polypeptides have the advantage in that they can be administered directly to an individual, or they can be expressed via an encoding nucleic acid by non-target cells, and they will travel to and concentrate in the CNS.

In one embodiment, the invention involves the treatment of neuronal disorders through gene delivery of a therapeutic polypeptide to an unaffected cell type. The unaffected cell type or non-target cell is used as a producer of the therapeutic polypeptide to secret effective amounts for vascular delivery to target cells. The therapeutic polypeptide contains, for example, a BBB-targeting moiety that facilitates concentration and translocation across the BBB. Once across, the therapeutic polypeptide can perform its function within the CNS cellular environment. In another embodiment, the CNS targeting moiety doubled as a lysosomal targeting moiety that further allowed concentration within the lysosomes of neuronal cells for the treatment of lysosomal storage disorders within the CNS.

As used herein, the term “chimeric” when used in reference to a central nervous system (CNS) targeting polypeptide of the invention is intended to mean a polypeptide composed of two or more heterologous polypeptide sequences fused together into a single primary amino acid sequence. Joinder of two or more heterologous amino acid sequences can be performed by, for example, chemical, biochemical or recombinant means. A chimeric polypeptide can therefore include, for example, a recombinant fusion protein or a chemical conjugate as well as other molecular complexes well known to those skilled in the art. When used in reference to a CNS targeting polypeptide, a chimeric polypeptide can be composed of, for example, a BBB-receptor binding domain derived from one molecule and a payload polypeptide domain derived from a different molecule. Both portions of the chimeric polypeptide can be derived from the same or a different species, including human, for example. Various other examples of chimeric polypeptides are well known to those skilled in the art and are included within the meaning of the term as it is used herein.

As used herein, the term “targeting polypeptide” when used in reference to a chimeric polypeptide is intended to mean a polypeptide that contains a binding partner to a molecule expressed on the surface of a targeted cell or tissue, or to a molecule that is otherwise accessible to the targeting polypeptide. Fusion of a binding partner recognized by a targeted cell receptor or ligand, for example, to a payload polypeptide domain allows the payload polypeptide to be directed to and bind to a predetermined target cell or tissue type. A targeting polypeptide can consist of, or include, any molecule that exhibits binding affinity toward a cognate binding partner. When used in reference to a polypeptide that targets CNS cells or tissues, a targeting polypeptide will include a binding partner recognized by CNS cells or tissues, including for example, cells constituting the blood-brain barrier (BBB). Therefore, a chimeric CNS targeting polypeptide can include, for example, a ligand, receptor, co-receptor, counter-ligand, counter-receptor, antigen or epitope, or a binding fragment thereof, as well as other affinity binders well known to those skilled in the art.

As used herein, the term “blood-brain barrier-receptor” or “BBB-receptor” when used in reference to binding domain is intended to mean the active binding portion of a ligand or receptor that is bound by a BBB-receptor. Use of the terms “ligand” or “receptor” refers to a molecule that exhibits selective binding affinity for another molecule. A ligand or a receptor is one component of a bi- or multi-component affinity binding reaction. As one constituent of two or more interacting molecular binding species, reference to a ligand or a receptor as a BBB-receptor binding domain is intended to be neutral with reference to binding partner orientation. Therefore, reference to a ligand or to a receptor as a BBB-receptor binding domain can refer to all types of affinity ligands well known to those skilled in the art including, for example, ligands, haptens, counter-ligands, receptors and counter-receptors. For simplicity, and where clarity may be desired when referring to both or all components of a BBB-receptor binding reaction, reference may be made to one component as a binding domain or ligand and to the cognate component as a BBB-receptor, receptor or counter-ligand. However, it should be understood that just as a ligand can be referred to equally as either a ligand or a receptor so can a BBB-receptor binding domain. Other nomenclature well known to those skilled in the art which designates one partner of a pair or complex of affinity binding components is included within the meaning of the term as it is used herein.

Affinity binding of a BBB-receptor binding domain can be, for example, through non-covalent or covalent interactions. BBB-receptor binding domains can include a wide range of molecular species including, for example, BBB-receptor binding polypeptides and functional fragments thereof. Specific examples of BBB-receptor binding domains include, for example, the ApoB polypeptide fragments described herein that bind to megalin and low-density lipoprotein receptor (LDLR); the ApoE polypeptide fragments described herein that bind to megalin, apolipoprotein E receptor 2, low-density lipoprotein related receptor (LRP), very-low density lipoprotein receptor (VLDL-R) and LDLR; the polypeptide fragments of aprotinin, lipoprotein lipase, α2-macroglobulin (α2M), PAI-I and pseudomonas exotoxin A described herein that bind to LDLR.

As used herein, the term “payload polypeptide domain” or “payload” is intended to mean the polypeptide portion connected to a BBB-receptor binding domain that is related to the purpose of a delivered targeting polypeptide. A payload polypeptide domain is distinguishable from a BBB-receptor binding domain because the latter functions in the delivery operation of the targeting polypeptide. In general, a payload polypeptide domain is an amino acid sequence that is connected to a BBB-receptor binding domain in a location other than its biologically active region or regions. For example, a payload binding domain can be attached to a BBB-receptor binding domain at amino acid residues outside of its enzymatic active site or receptor binding domain. A payload polypeptide domain can be fused to, for example, the amino-terminal, carboxyl-terminal or both termini of a BBB-receptor binding domain. Accordingly, a payload polypeptide domain of the invention is a polypeptide that is targeted by a BBB-receptor binding domain of the invention.

As used herein, the term “functional fragment” when used in reference to a BBB-receptor binding domain or in reference to a payload polypeptide domain is intended to mean a portion of a BBB-receptor binding domain which retains some or all of the selective binding of the intact BBB-receptor binding polypeptide or a portion of a payload polypeptide domain which retains some or all of the selective enzymatic, structural or other biochemical activity of the intact payload polypeptide. Such functional fragments can include, for example, truncated, deleted or substituted amino acid residues of the intact or parent polypeptide so long as it retains some selective binding or activity as exhibited by the larger parent BBB-receptor binding polypeptide or the larger parent payload polypeptide. Specific examples of a functional fragment of a BBB-receptor binding domain include the ApoB, ApoE, aprotinin, lipoprotein lipase, α2-macroglobulin (α2M), PAI-I and pseudomonas exotoxin A polypeptide fragments described herein as well as other polypeptide fragments described further below and those polypeptide fragments well known to those skilled in the art. Specific examples of a functional fragment of a payload polypeptide include the active site domains for any of the therapeutic polypeptides described herein as involved in mucopolysaccharidoses, Fabry disease, Schnidler disease, Alzheimer's, Tay-Sachs, Parkinson's or other neural degenerative disorders, neuropathologies or other CNS-associated disorders. Binding activity of functional fragments can be retained, for example, where the three dimensional structure of the parent polypeptide framework is substantially retained.

BBB-receptor binding domains, payload polypeptide domains or functional fragments thereof are intended to include amino acid sequences having minor modifications of a parent polypeptide amino acid sequence but which exhibits some or all of the selective binding of the intact BBB-receptor binding polypeptide or a portion of a payload polypeptide domain which retains some or all of the selective enzymatic, structural or other biochemical activity of the intact payload polypeptide. Minor modifications of polypeptides having selective binding or activity as the parent polypeptide include, for example, conservative substitutions of naturally occurring amino acids and as well as structural alterations which incorporate non naturally occurring amino acids, amino acid analogs and functional mimetics.

For example, Arginine (Arg) is considered to be a conservative substitution for the amino acid Lysine (Lys). Other conservative amino acid substitutions and functional equivalents are well know in the art and can be found described in, for example, in Lehninger Principles of Biochemistry, Nelson and Cox, Third Edition, 2000, Worth Publishers, New York and Biochemistry, Stryer, Fourth Edition, 1995, W.H. Freeman and Company, New York. Similarly, mimetic structures substituting positive or negative charged or neutral amino acids, with organic structures having similar charge and spacial arrangements also are considered a functional equivalent of a parent amino acid sequence so long as the polypeptide mimetic exhibits selective binding or activity as the parent polypeptide. Given the teachings and guidance provided herein, those skilled in the art will known, or can determine, which conservative substitutions, amino acid analogs, or functional mimetic structures will function as an equivalent of a BBB-receptor binding domain or of a payload polypeptide domain or as an amino acid residue thereof.

As used herein, the term “effective amount” when used in reference to administration of a chimeric CNS targeting polypeptide, encoding nucleic acid or a vector containing such a polypeptide or encoding nucleic acid is intended to mean an amount of such a molecule or particle required to effect a beneficial change in a clinical symptom, physiological state or biochemical activity targeted by a chimeric CNS targeting polypeptide of the invention. For example, for the therapeutic payload polypeptide domains that can be used in the methods of the invention, an effective is an amount sufficient to decrease the extent, amount or rate of progression of the targeted pathological condition. The dosage of a chimeric CNS targeting polypeptide, encoding nucleic acid or vector particle required to be therapeutically effective will depend, for example, on the neurological or other CNS disease to be treated, the route and form of administration, the potency and bio active half life of the molecule being administered, the weight and condition of the individual, and previous or concurrent therapies. The appropriate amount considered to be an effective dose for a particular application of the method can be determined by those skilled in the art, using the teachings and guidance provided herein. For example, the amount can be extrapolated from in vitro or in vivo assays or results from clinical trials employing related or different therapeutic molecules or treatment regimes. Those skilled in the art will recognize that the condition of the patient can be monitored, for example, throughout the course of therapy and that the amount of the chimeric CNS targeting polypeptide that is administered can be adjusted accordingly.

As used herein, the term “depot” when used in reference to administration of a chimeric CNS targeting polypeptide is intended to mean a cell or population of cells that produce a referenced chimeric CNS targeting polypeptide of the invention. A depot cell therefore acts as an in vivo polypeptide factory to produce a chimeric CNS targeting polypeptide. The produced chimeric CNS targeting polypeptides can be secreted, for example, into the blood steam, body fluids or surrounding tissues where they can act on proximal or distal cells. Transfer and concentration of chimeric CNS targeting polypeptides to distal locations within a tissue or organism is accomplished via a targeting domain such as a BBB-receptor binding domain. The chimeric CNS targeting polypeptides can be produced by, for example, expression or expression and secretion of an encoding nucleic acid. A producer cell can be, for example, a non-targeted cell type for expression and delivery to proximal or distal cell types or a targeted cell type for expression and delivery to, for example, proximal cell types. A depot cell will generally be, for example, a non-CNS cell type which is accessible for in vivo or in vitro genetic modification by an encoding nucleic acid. A depot cell can therefore effect the expression, secretion and diffusion of a chimeric CNS targeting polypeptide capable of transversing the BBB.

The invention provides a chimeric CNS targeting polypeptide having a BBB-receptor binding domain and a payload polypeptide domain. The BBB-receptor binding domain can be a receptor binding domain derived from ApoB, ApoE, aprotinin, lipoprotein lipase, PAI-1, pseudomonas exotoxin A, transferrin, α2-macroglobulin, insulin-like growth factor or insulin, or a functional fragment from any of these BBB-receptor binding polypeptides.

Lysosomal enzymes are expressed constitutively in all cells of the body. Messenger RNA is translated and translocated into the endoplasmic reticulum (ER) upon which secretory polypeptides undergo high mannose N-linked glycosylation. Glycosylated lysosomal enzymes are recognized and phosphorylated at the terminal mannose residues. These phosphorylated mannose residues are recognized by the resident ER receptor Mannose 6-Phosphate Receptor (M6P) and shuttled to the lysosome. M6P receptors are localized to the ER and the plasma membrane where they can capture lysosomal enzymes from the blood stream and transport them to the lysosome. Harnessing this lysosomal polypeptide and receptor cyclization pathway, expression of a lysosomal enzyme from one cell can provide the polypeptide to surrounding cells so that cross-correction of a large number of cells can be achieved by delivering the gene for a deficient lysosomal enzyme to a few widely scattered cells. The harnessing of the lysosomal cyclization pathway can occur, for example, in conjunction with a chimeric CNS targeting polypeptide that first targets the payload polypeptide across the BBB. Alternatively, it can be harnessed in connection with non-CNS targeting domains to deliver a payload polypeptide to non-CNS or peripheral locations of an organism, including a human.

Similarly, cross-correction of a sufficient number of cells also can be employed for targets of non-lysosomal related disorders where the therapeutic polypeptide has a cognate cell surface receptor that can be internalized or where another mechanism of cellular entry exists. Further, cross-correction by expression and secretion of a polypeptide can further be employed where the therapeutic polypeptide is required to supply an extracellular function. An efficacious feature in all of such treatments, whether direct enzyme or polypeptide replacement or whether replacement by in vivo expression and secretion, is the ability of the therapeutic polypeptide to be targeted to the defective cellular location. A second efficacious feature is the ability of the therapeutic polypeptide to be taken up by a defective cell where it has an intracellular function to perform.

An impediment to targeting therapeutic polypeptides to the CNS is the blood-brain barrier (BBB). As described previously, this tissue structure prevents polypeptides from diffusion into the CNS unless there is a specific receptor for that molecule. The invention provides targeting polypeptides that can be specifically translocated across the BBB for deposition into the vascular and other fluid systems of the CNS. The targeting polypeptides can contain, for example, additional functional domains that are chaperoned by the CNS targeting portion of the targeting polypeptide across the BBB and into the CNS. Once across, the CNS targeting polypeptides of the invention are free to perform the functions associated with them by attachment to the CNS targeting portion. Such functions can be, for example, therapeutic or diagnostic. The associated activities can include, for example, enzymatic, structural or binding activities.

The CNS targeting polypeptides of the invention include a chimeric polypeptide structure. The chimeric molecule contains at least a targeting domain for selective binding and translocation across the BBB. A receptor binding domain recognized by at least the BBB constitutes a targeting domain of a chimeric CNS targeting polypeptide of the invention. The targeting domain also can be recognized by cells or structures within the CNS.

A targeting domain recognized by the BBB can be, for example, a BBB-receptor binding domain. A BBB-receptor binding domain can be derived from any polypeptide or other molecule that selectively binds to a receptor within the BBB. Such BBB-receptor binding domains can constitute, for example, an intact ligand or polypeptide that is selectively bound by a BBB-receptor. Alternatively, a BBB-receptor binding domain can be, for example, an functional fragment of such BBB-receptor binding domains. Specific examples of BBB-receptor binding domains include, for example, the polypeptides or their receptor binding domains from ApoB, ApoE, aprotinin, lipoprotein lipase, PAI-1, pseudomonas exotoxin A, transferrin, α2-macroglobulin, insulin-like growth factor or insulin.

For example, ApoB and the ApoB polypeptide fragments described herein bind to the BBB-receptors megalin and low-density lipoprotein receptor (LDLR). ApoE and the ApoE polypeptide fragments described herein bind to megalin, apolipoprotein E receptor 2, low-density lipoprotein related receptor (LRP), very-low density lipoprotein receptor (VLDL-R) and LDLR. Aprotinin, lipoprotein lipase, α2-macroglobulin (α2M), PAI-I and pseudomonas exotoxin A and their respective polypeptide fragments described herein bind to LDLR. A specific example of an ApoB fragment constituting a BBB-receptor binding domain is the amino acid sequence PSSVIDALQYKLEGTTRLTRKRGLKLATALSLSNKFVEGSPS (SEQ ID NO: 1). A specific example of an ApoE fragment constituting a BBB-receptor binding domain is the amino acid sequence VDRVRLASHLRKLRKRLLR (SEQ ID NO: 2). Both of these BBB-receptor binding domains selectively bind, for example, LDLR. A specific example of an aprotinin fragment constituting a BBB-receptor binding domain is the amino acid sequence RRPDFCLEPPYTGPCKARIIRYFYNAKAGLCQTFVYGGCRAKRNNFKSAEDCMRTCGG A (SEQ ID NO: 3), which binds the megalin receptor, for example. Accordingly, functional fragments of BBB-receptor binding polypeptides or domains also can be used as a targeting moiety for the chimeric CNS targeting polypeptides of the invention.

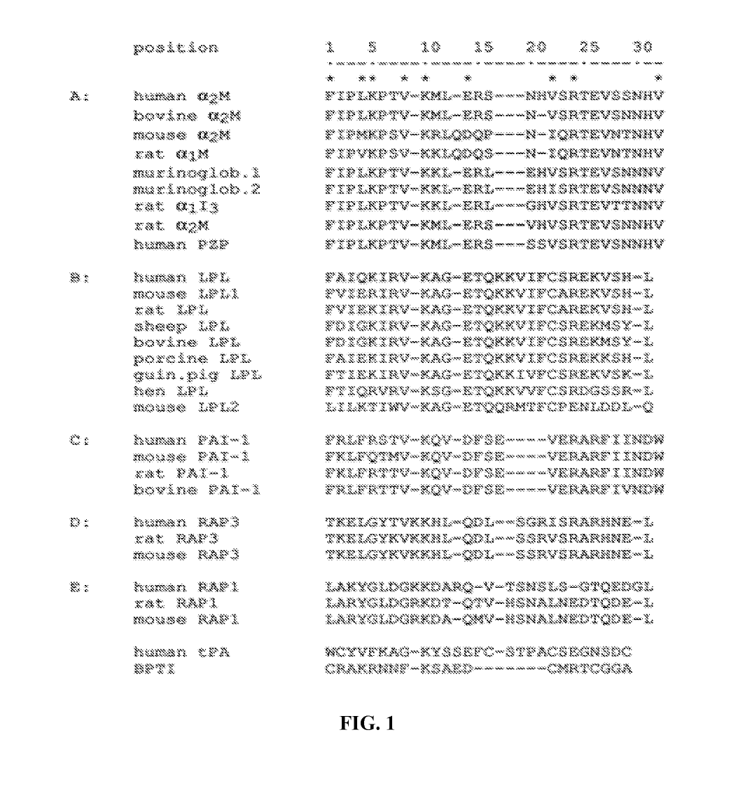

Other polypeptides recognized by a BBB-receptor that can be used as a targeting component of a chimeric CNS targeting polypeptide of the invention include, for example, transferrin, angiotensin II, arginine vasopressin, atrial natriuretc peptide, brakykinin, brain natriuretic peptide, endothelin, insulin like growth factors, insulin, neuropeptide Y, oxytocin, pancreatic polypeptide, prolactin, somatostatin, substance P and vasoactive intestinal polypeptide as well as those amino acid sequences and their corresponding parent polypeptides listed in FIG. 1. Additionally, the BBB-receptor binding domain of these polypeptides also can be removed from the parent polypeptide framework and employed as a targeting component of the chimeric CNS targeting polypeptide of the invention. A description of the receptor binding activity of the above described polypeptides can be found described in, for example, Moos and Morgan, Cell. & Mol. Neurobio., 20:77-95 (2000); Nielsen et al., J. Biol. Chem., 271:12909-12 (1996); Kounnas et al., J. Biol. Chem., 267:12420-23 (1992); Moestrup et al., J. Clin. Invest., 96:1404-13 (1995); Norris et al., Biol. Chem. Hoppe Seyler, 371 Suppl:37-42 (1990), and Ermisch et. al., Phys. Revs. 73:480-527 (1993).

Polypeptides, or their functional fragments, that are known to cross the BBB can similarly be employed as a targeting component of a chimeric CNS targeting polypeptide. Translocation of such polypeptides across the BBB indicates the existence of a cognate receptor binding partner to the translocated ligand. Accordingly, these polypeptides or their BBB-receptor binding domains, as well as other polypeptides known in the art which can cross the BBB, can be employed as a BBB-receptor binding domain of the chimeric polypeptides of the invention even in the absence of an identified cognate receptor. Specific examples of such BBB-translocating polypeptides include α MSH, adrenocorticotropin analogues, β casomorphin, β endorphin and analogues, bovine adrenal medulla dodecapeptide, corticotropin releasing hormone, cyclo Leu Gly (diketopiperazine), D Ala peptide T amide, delta sleep inducing peptide, encaphalins and analogues, FMRF, gastrin releasing peptide, glucagon, growth hormone releasing hormone, insulin, luteinizing hormone releasing hormone (GnRH), oxytocin, Pro Leu Gly (MIF 1 MSH release inhibiting factor), prolactin, somatostatin and analogues, substance P, thyrotropin releasing hormone (TRH), Tyr MIF 1. A description of the BBB translocation activity of these polypeptides can be found described in, for example, Banks and Kastin. “Bidirectional passage of peptides across the blood brain barrier.” In Circumventricular Organs and Brain Fluid Environment; A. Ermisch, R. Landgraf & H J. Rühle, Eds. Prog. Brain Res. 91:139-148 (1992), and Begley, D. J., “Peptides and the blood brain barrier.” In Handbook of Experimental Pharmacology: Physiology and Pharmacology of the Blood Brain Barrier. M.W.B. Bradbury, Ed. Vol. 103:151-203. Springer, Berlin, (1992).

Those skilled in the art will known, or can determine, which amino acid residues of a BBB-receptor binding polypeptide constitute a functional fragment sufficient to selective bind a BBB-receptor. For example, it is routine to make and test successively smaller polypeptide fragments, either recombinantly or chemically, and test them for binding activity. Therefore, any of the BBB-receptor binding polypeptides described above, or portions thereof corresponding to a BBB-receptor binding domain, can be used as a CNS targeting component in a chimeric CNS targeting polypeptide of the invention. Other BBB-receptor binding polypeptides know to those skilled in the art can similarly be used as a CNS targeting component in a chimeric CNS targeting polypeptide of the invention.

The choice of BBB-receptor binding domain will depend on the receptors available within the BBB that can be targeted and utilized for binding and translocation of a targeting polypeptide into the CNS. Essentially, any BBB-receptor binding polypeptide or BBB-receptor binding domain can be incorporated into a chimeric CNS targeting polypeptide so long as a cognate receptor is located in the BBB. Receptors useful in targeting a chimeric CNS targeting polypeptide of the invention include those receptors that bind to ApoB, ApoE, aprotinin, lipoprotein lipase, α2-macroglobulin (α2M), PAI-I and pseudomonas exotoxin A, as described above. Briefly, such receptors include, for example, LDLR, megalin, apolipoprotein E receptor 2 (ER2), LRP, VLDL-R and LDLR.

Other receptors available for targeting with a cognate binding partner such as a ligand include, for example, transferrin, angiotensin II, arginine vasopressin, atrial natriuretc peptide, brakykinin, brain natriuretic peptide, endothelin, insulin like growth factors, insulin, neuropeptide Y, oxytocin, pancreatic polypeptide, prolactin, somatostatin, substance P and vasoactive intestinal polypeptide as well as receptors to the parent polypeptides set forth in FIG. 1 and the BBB-translocating polypeptides described previously. By similar analogy, for targeting of chimeric polypeptides to cells or tissue other than the CNS, it is sufficient to have a targeting domain selective for the targeted cell type or tissue in order to allow concentration through binding of the target receptor binding domain to its cognate receptor on a target cell.

The chimeric CNS targeting polypeptides of the invention also contain at least a payload polypeptide domain for delivery to a targeted location and execution of a desired function. The function can include, for example, enzymatic, structural or binding or any combination thereof. Therefore, a payload polypeptide domain can be any polypeptide that is desirable to deliver to a target site.

Desirable polypeptides to deliver to a specific location within an organism or tissue will depend on, for example, the function sought to be replaced or supplemented. For example, in lysosomal storage disorders, a payload polypeptide corresponding to the defective lysosomal enzyme will be desirable. In neuronal degenerative diseases, for example, a payload polypeptide having a defective activity causative or contributory to the degenerative disease will be desirable to deliver to CNS cells. Similarly, in other neuronal pathologies a payload polypeptide having an activity that is corrective or beneficial to the clinical symptoms will be desirable to deliver using a chimeric CNS targeting polypeptide of the invention. Neuronal proliferative diseases similarly can be treated using a chimeric CNS targeting polypeptide of the invention by, for example, delivering a polypeptide having an activity that retards cell proliferation or results in loss of viability. Similarly, payload polypeptides for the treatment of proliferative disorders that can induce programmed cell death also can be used. Those skilled in the art will known what polypeptide or functional fragment thereof can be used to treat a particular disorder or to augment or supplement treatment of a disorder given the teachings and guidance provided herein.

For the specific example of lysosomal storage disorders, a payload polypeptide can include any of the deficient lysosomal or polypeptide activities associated with such disorders. Specific lysosomal storage disorders include, for example, mucopolysaccharidoses, Krabbe disease, metachromatic leukodystrophy, Fabry disease and Schnidler disease. Briefly, MPSI is defective in α-L-iduronidase activity; MPSII is defective in iduronate sulfatase activity; MPSIIIa is defective in heparan N-sulfatase activity; MPSIIIb is defective in α-N-acetylglucosaminidase activity; MPSIIIc is defective in actelyl-CoA:α-glucosaminide acetyltransferase activity; MPSIIId is defective in N-aceteylglucosamine 6-sulfatase activity; MPSIVa is defective in galactose 6-sulfatase activity; MPSIVb is defective in β-galactosidase activity; MPSVI is defective in N-acetylgalactosamine 4-sulfatase activity; MPSVII is defective in β-glucuronidase activity; Krabbe disease is defective in galactocerebroside β-galactosidase activity or β-glucocerebrosidase; metachromatic leukodystrophy is defective in arylsulfatase A, arylsulfatase B or arylsulfatase C; Frabry disease is defective in α-galactosidase activity and Schnidler disease is α-N-acetylgalactosaminidase activity.

With regard to other neuronal disorders and CNS pathologies, Alzheimer's disease is defective in β-amyloid endopeptidase activity; Tay-Sachs disease is defective in hexosaminidase α-subunit or hexosaminidase β-subunit activity and Parkinson's disease as well as other neuronal degenerative disorders are defective in neural growth factors, for example. Other neuronal disorders and pathologies and their associated defective polypeptide activity are well known to those skilled in the art.

A payload polypeptide domain for targeted delivery to the CNS for the treatment of any of the above diseases can exhibit the functional activity of the defective enzyme. Therefore, for the above lysosomal storage diseases, neuronal degenerative disorders and other neuronal disorders or pathologies, a payload polypeptide can be, for example, α-L-iduronidase, iduronate sulfatase, heparan N-sulfatase, α-N-acetylglucosaminidase, actelyl-CoA:α-glucosaminide acetyltransferase, N-aceteylglucosamine 6-sulfatase, galactose 6-sulfatase, β-galactosidase, N-acetylgalactosamine 4-sulfatase, β-glucuronidase, galactocerebroside β-galactosidase, β-glucocerebrosidase, arylsulfatase A, arylsulfatase B, arylsulfatase C, α-galactosidase, α-N-acetylgalactosaminidase, endopeptidase, hexosaminidase α-subunit, hexosaminidase β-subunit or a neural growth factor, or a functional fragment thereof.

Construction of a chimeric CNS targeting polypeptide of the invention can be performed by any method well known to those skilled in the art. For example, a chimeric CNS targeting polypeptide can be generated by recombinant methodology, including for example, in vitro or in vivo expression as well as by chemical synthesis. Such methods can be found described in, for example, Sambrook et al., Molecular Cloning: A Laboratory Manual, Cold Spring Harbor Laboratory, New York (1992) and in Ausubel et al., Current Protocols in Molecular Biology, John Wiley and Sons, Baltimore, Md. (1989). The location of a BBB-receptor binding domain relative to the payload polypeptide domain will generally be at a termini, such as at the amino- or carboxyl-terminus of the payload polypeptide amino acid sequence. However, it should be understood that any location will suffice so long as the function of each component of the chimeric targeting polypeptide is retained. Accordingly, a BBB-receptor binding domain also can be internal to a terminus of a payload polypeptide domain so long as the selective binding function of the targeting domain is retained and so long as the activity of the payload polypeptide is retained. Those skilled in the art will know, or can determine, what orientations and locations are amenable to a particular application and for a particular BBB-receptor binding domain and payload polypeptide domain pair.

The chimeric CNS targeting polypeptide of the invention can further include any of a variety of other moieties that are beneficial for the targeting operation or for the intended functional result. For example, and as described further below in reference to the methods of the invention, a chimeric CNS targeting polypeptide can further include a secretory signal. The secretory signal can specify intracellular trafficking of the polypeptide or it can specify secretion into the vascular or other extracellular fluid or space. A specific example of a secretory signal can be, for example, pre-pro trypsin secretory signal or other secretory signal well known to those skilled in the art.

Other moieties that can be incorporated within a chimeric CNS targeting polypeptide of the invention include, for example, a tag. Such tags include, for example, molecular tags that can be used in the detection or isolation of the chimeric CNS targeting polypeptide. Such tags can function in detection or isolation using, for example, fluorescent, affinity or enzymatic methods. Specific examples of such tags include, for example, green fluorescent protein or an epitope tag such as myc. Other methods of detection and modes of isolation well known in the art can similarly be employed with a corresponding tag using the teachings and guidance provided herein.

Although the invention is described above and below with reference to polypeptides that target the BBB for translocation and delivery to cells of the CNS, those skilled in the art will understand given the teachings and guidance provided herein that the chimeric targeting polypeptides and the methods of targeting are equally applicable to delivery of payload polypeptide domains, for example, to cells other than the CNS. Similarly, the chimeric targeting polypeptides and methods described herein also can be used for multiple, step-wise, consecutive or simultaneous targeting to specific cells or tissue locations within the CNS or other parts of an organism. All that is sufficient for such targeting to other locations or to specific cells within the CNS is that the chimeric polypeptide contain a selective receptor binding domain that is present or available on the targeted cell type. Similarly, for the targeting of subcellular locations or organelles, all that is sufficient is the presence of a targeting domain for internalization and localization to the subcellular location or organelle. A specific example of subcellular organelle localization is the targeting of a lysosomal enzyme to the lysosomes within cells of the CNS as described herein. A specific example of a subcellular location is the targeted entry of a payload polypeptide to the cytoplasm as described previously. Targeting domains for other subcellular locations or organelles are well known to those skilled in the art and can be employed in the chimeric targeting polypeptides and method of the invention given the teachings and guidance provided herein.

The invention also provides a nucleic acid encoding a chimeric CNS targeting polypeptide having a nucleotide sequence encoding a BBB-receptor binding domain and a nucleotide sequence encoding a payload polypeptide domain. Nucleotide sequences encoding chimeric CNS targeting polypeptides described above can be determined based on the information contained within the genetic code. Nucleic acids can be chemically synthesized or produced by recombinant methods well known to those skilled in the art. Such methods can be found described in, for example, Sambrook et al., supra, and Ausubel et al., supra, and the references cited therein. Accordingly, a nucleic acid encoding any desired combination of BBB-receptor binding domain and payload polypeptide domain can be routinely constructed. Such encoding nucleic acids are useful, for example, in the in vivo or in vitro production of chimeric CNS targeting polypeptides of the invention.

The invention also provides a method of delivering a polypeptide to the CNS of an individual. The method consists of administering to an individual an effective amount of a chimeric CNS targeting polypeptide, the chimeric CNS targeting polypeptide having a BBB-receptor binding domain and a payload polypeptide domain.

As described previously, BBB-receptors include, for example, low-density lipoprotein receptor, transferrin receptor, insulin receptor and insulin growth factor receptor. The BBB translocation function of these and other BBB-receptors can be harnessed for CNS targeting of a payload polypeptide.

Briefly, the low-density lipoprotein receptor family is a group of cell surface receptors that bind lipoprotein complexes for internalization to the lysosomes. The family comprises approximately ten different receptors with the most common examples being low-density lipoprotein receptor (LDLR), low-density lipoprotein related receptor (LRP), very-low density lipoprotein receptor (VLDL), megalin and apolipoprotein E receptor 2. The receptors are expressed in a tissue specific manner and primarily bind apolipoprotein complexes. The apolipoprotein, of which the two most prominent members are apolipoprotein B (ApoB) and apolipoprotein E (ApoE), function to bind lipids in the blood stream and target them for lysosomal degradation. Binding of the apolipoproteins to the receptor results in endocytosis and transport to the lysosome where the low pH compartment facilitates the release of the polypeptide complex. The LDL receptor is then recycled to the cell surface. At the blood brain barrier, the LDL receptor binds lipoproteins resulting in endocytosis. Rather than transport to the lysosome, the LDL receptor is shuttled to the apical side of the BBB where presumably, the apolipoprotein is released to be taken up by neurons and/or astrocytes.

The ability of chimeric CNS targeting polypeptides of the invention to bind to a BBB-receptor or other targeted receptor endows them with the quality to concentrate or home to the location of such receptors. Concentration occurs by, for example, diffusion, passive transportation via blood or other bodily fluids or other physiological mechanisms through the body until a receptor binding domain come in contact with its cognate receptor or counter-ligand. Once in contact, binding and retention occurs at the site of the targeted receptor, thereby producing a sink which effectively concentrates the chimeric targeting polypeptide. Therefore, the chimeric targeting polypeptides of the invention can be used in polypeptide replacement therapy or diagnostic procedures for the delivery of a desirable payload polypeptide to both CNS and non-CNS target cells alike.

An effective amount of the chimeric targeting polypeptides is administered to carry out the function of the targeting domain and the payload domain. An effective amount for targeted therapeutic treatments or diagnostic applications effective amount of a chimeric CNS targeting polypeptide, or corresponding molar equivalent of either a BBB-receptor binding domain or a payload polypeptide domain, can be, for example, between about 10 μg/kg to 500 mg/kg body weight, for example, between about 0.1 mg/kg to 100 mg/kg, or preferably between about 1 mg/kg to 50 mg/kg, depending on the treatment regimen. For example, if a chimeric CNS targeting polypeptide is administered from one to several times a day, or by low in vivo expression, then a lower dose would be needed than if a chimeric CNS targeting polypeptide were administered weekly, monthly or less frequently, or by high, constitutive in vivo expression methods. Similarly, formulations that allow for timed release or regulated in vivo expression of a chimeric CNS targeting polypeptide would provide for the continuous release of a smaller amount of a chimeric targeting polypeptide than would be administered as a single bolus dose. For example, a chimeric CNS targeting polypeptide can be administered by in vivo expression or by methods of infusion at 4 mg/kg/week.

For CNS targeted delivery, a chimeric CNS targeting polypeptide must first be targeted and transverse the BBB. Once across the BBB, a chimeric CNS targeting polypeptide will be available to supplement all cell types of the CNS. For targeting to a specific CNS cell type, cytoplasmic internalization or to lysosomal or other subcellular organelles, a chimeric CNS targeting polypeptide can contain, for example, an additional targeting moiety to effect this desired result. In the specific case of lysosomal targeting, certain BBB-receptor binding domains, as described previously, simultaneously confer both BBB-receptor targeting and subcellular internalization and lysosomal targeting because the same receptor binding specificity is present on both cells of the BBB and cells within the CNS. In contrast, for non-CNS targeted delivery, it is sufficient for a chimeric targeting polypeptide to contain a targeting receptor binding domain selective for the ultimate non-CNS target cell type.

Delivery of a chimeric CNS targeting polypeptide or other chimeric targeting polypeptide can occur by various modes of administration well known to those skilled in the art. As described above, because the chimeric targeting polypeptides of the invention are endowed with the ability to concentrate at the targeted site due to its selective binding characteristics, essentially any mode of delivery of the chimeric targeting polypeptides of the invention to an individual will achieve this outcome. For example, a chimeric CNS targeting polypeptide can be injected or infused into an individual for diffusion and binding, for example, at the BBB and subsequent translocation across this CNS barrier. Those skilled in the art will understand that delivery by injection or infusion can require repeated administrations to maintain an effective amount for therapeutic treatment.

A chimeric targeting polypeptide can be delivered systemically, such as intravenously or intraarterially. A chimeric CNS targeting polypeptide also can be administered locally at a site of a depot producer cell. Appropriate sites for administration of chimeric polypeptide are known or can be determined by those skilled in the art depending on the clinical indications of the individual being treated. For example, the chimeric CNS targeting polypeptide described above can be provided as isolated and substantially purified polypeptides in pharmaceutically acceptable formulations using formulation methods known to those of ordinary skill in the art. These formulations can be administered by standard routes, including for example, topical, transdermal, intraperitoneal, intracranial, intracerebroventricular, intracerebral, intravaginal, intrauterine, oral, rectal or parenteral (e.g., intravenous, intraspinal, subcutaneous or intramuscular) routes. Osmotic minipumps can also be used to provide controlled delivery of high concentrations through cannulae to the site of interest, such as directly into a depot organ or into the vascular supply.

Alternatively, a chimeric CNS targeting polypeptide or other chimeric targeting polypeptide of the invention can be administered by cell therapy with cells engineered to express such targeting polypeptides. Cell therapy can include, for example, the transplantation or implantation of such engineered cells under conditions that maintain viability of the modified cells. Transplantation can occur with solid tissues as well as with bone marrow or other hematopoietic cell types. Solid tissues can include, for example, liver, fibroblasts and other tissues or cell types found within an organism, including a human individual. Methods for cell therapy, including transplantation and implantation, of a variety of cell and tissue types are well known to those skilled in the art. Such methods can be routinely implemented with cells genetically modified to express a chimeric CNS targeting polypeptide or other chimeric targeting polypeptide of the invention.

Administration also can be by gene delivery of an encoding nucleic acid. Gene delivery can be effected by a variety of methods well know to those skilled in the art. An encoding nucleic acid for a chimeric CNS targeting polypeptide or other targeting polypeptide can be incorporated into a nucleic acid vector or a viral vector and delivered to depot cells for synthesis and secretion into the blood or other bodily fluids of the individual.

For example, encoding nucleic acids can be delivered to a depot organ by injection of naked nucleic acid into muscle, skin or other accessible organs. Additionally, the encoding nucleic acids can be delivered to a depot organ using, for example, a targeting viral, liposome or other particle vector. Typical viral vectors include lentiviral viral vectors, adenoviral vectors, retroviral vectors, oncoretroviral vectors, such as the Moloney leukemia virus (MLV) as well as other DNA or RNA viral vectors. Methods for constructing and using such viral vectors are well known in the art. Additionally, viral vectors have the advantage of being amenable to alter target specificity by appropriate pseudotyping of the viral particle. Using well known pseudotyping methods, those skilled in the art can produce a wide variety of viral vector particles harboring a nucleic acid encoding chimeric CNS targeting polypeptide or other chimeric targeting polypeptide of the invention.

A particularly useful viral vector is the lentiviral vector. The design of a viral vector system for therapeutic or diagnostic gene delivery can be based on the segregation of the viral genome of cis acting sequences involved in its transfer to target cells from trans acting sequences encoding the viral polypeptides. The vector particle is assembled by viral polypeptides expressed from nucleic acid constructs stripped of cis acting sequences. The cis sequences are instead incorporated into a nucleic acid vector for expression of the transgene to create the vector's genome. This vector genome, or transducing vector, is endowed with a full complement of cis acting sequences which allows its encapsidation and transfer to the target cell. Because the particle will transfer only the vector genome, the target cell will be devoid of trans-acting polypeptides needed for further vector particle production and the infection process is limited to a single round without spreading. By separating the cis- and trans-acting viral functions, a safe and efficient lentiviral vector system can be produced. For the particular use of lentiviral-based vectors in therapeutic or diagnostic applications, it can be desirable to separate many, if not all of the cis- and trans-acting functions, of the vector genome from the packaging system nucleic acid constructs.

Several cis sequences have been implicated in the encapsidation and dimerization of lentiviral viral RNA. For example, the packaging signal or Ψ sequence, located in the untranslated leader downstream of the major splice donor site, contributes to RNA packaging and discrimination of genomic from spliced transcripts. Additional sequences contributing to encapsidation and genome discrimination have been identified in the transcribed long terminal repeats (LTR) and 59 nucleotide (nt) leader sequence upstream of the major splice donor site. Lentiviral packaging signal sequences can be found described in, for example, Lever et al., J. Virol. 70:721-28 (1989); Aldovini and Young, J. Virol. 63:1920-26 (1990); Luban et al., J. Virol. 68:3784-93 (1994); Kim et al., Virology 198:336-40 (1994); Vicenzi et al, J. Virol. 68:7879-90 (1994); Geigenmuller et al., J. Virol. 70:667-71 (1996); Paillart et al., Proc. Natl. Acad. Sci. USA, 93:5572-77 (1996), and McBride and Panganiban, J. Virol. 70:2963-73 (1996). Therefore, depending on the desired efficiency, a lentiviral packaging signal included in a vector genome of the invention can be, for example, a lentiviral Ψ sequence alone or a multipartite signal consisting of a Ψ sequence together with packaging determinants within its transcribed LTR leader sequence.

Features of the lentiviral packaging constructs that prevent their transfer to target cells include a several modifications to the viral sequence. Modifications at the 5′ end of the viral genome delete or disrupt structural motifs implicated in RNA encapsidation and dimerization. For example, deletion of the 5′ leader sequence reduces the encapsidation efficiency of lentiviral transcripts whereas removal of both LTRs and of the primer binding site from the packaging construct prevents reverse transcription and integration of any encapsidated transcript. The complement of gene product functions that can be included in a packaging construct or system can range from those lentiviral gene products necessary to achieve encapsidation to the full repertoire of trans-acting functions encoded in a lentiviral genome.

One mode of the packaging constructs and systems of the invention precludes the generation of replication-competent HIV viruses, even by unlikely rearrangement and recombination events because of the actual absence of most of HIV env sequences in any of the packaging constructs or vector genomes. The use of a separate construct encoding a heterologous targeting polypeptide, or an additional envelope polypeptide, makes it unlikely that a replication competent recombinant be generated. This unlikely event would require multiple recombination events between different construct plasmids and/or endogenous retroviral sequences, including recombination between nonhomologous sequences.

The lentiviral packaging constructs, systems and gene delivery systems incorporate the above-described considerations and functional requirements for component nucleic acid vectors needed to generate a vector of the invention. For production of a lentiviral vector of the invention, a lentiviral packaging construct can be generated which encodes trans-acting factors sufficient for lentiviral vector generation as described above and an attachment incompetent fusogenic polypeptide. Trans-acting factors sufficient for vector generation include, for example, the polypeptides encoded by the lentiviral gag, pol and rev genes. One or more of the lentiviral trans-acting factors can be encoded on a separate nucleic acid construct, such as a plasmid, such that the packaging construct consists of two or more plasmids. The separation of trans-acting factors onto separate plasmids further ensures against unwanted recombination events.

Infection of a target cell with a lentiviral vector is similar to a retroviral infection process. Once the content of a lentiviral vector is delivered inside the target cell, uncoating, reverse transcription, interaction with cytoplasmic chaperones and the nuclear import machinery, and maturation to an integration-competent complex takes place. The lentiviral vectors of the invention can therefore be used to transduce a cell with a transgene of interest. The lentiviral vectors of the invention also can be used to specifically target and deliver a transgene to a predetermined cell or tissue type. A lentiviral vector of the invention can function for either transduction or targeted transduction of a specific cell or tissue type. To effect transduction or targeting, the vector can contain a targeting polypeptide having a cognate binding partner on the cells to be transduced or targeted. The targeting polypeptide can be, for example, heterologous, chimeric or both. The various combinations and permutations of targeting polypeptides described previously are applicable to methods of using lentiviral vectors for specific, preferential or ubiquitous delivery of a therapeutic gene of interest.

For transduction of a cell or cell population is contacted with an effective amount of a lentiviral vector having incorporated into its envelope a fusogenic polypeptide and a heterologous targeting polypeptide which can bind to the cell or population. An effective amount is that amount sufficient for sufficient for vector binding and cell fusion. An effective amount of vector is between about 1ng-100 μg, generally, an effective amount is about 100ng-50 μg, and more generally an effective amount is about 1-100 μg. Conditions that are sufficient for transduction include essentially any physiologically compatible medium. Such conditions include, for example, cell culture medium and sterile physiological medium. Incubation times sufficient for transduction can range from about minutes, generally about 1-4 hrs, and more generally about 5-24 hrs. Other vector amounts and conditions sufficient for vector-cell fusion are well known to those skilled in the art and can similarly be used in the methods of the invention for transducing a cell or cell population using the lentiviral vectors of the invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Delivery of Glucocerebrosidase to the Liver and Brain for Treatment of Gaucher's Disease by Targeted Uptake via the LDL Receptor

Gaucher's disease is an inherited lysosomal storage disease resulting from mutations and loss of activity of glucocerebrosidase. Symptoms range from painful ‘bone crisis’ and hepatosplenomegaly to neurological disorders and death. There are no known effective treatments for the neurological disorders associated with the more severe Type 2 and Type 3 Gaucher's disease.

This Example shows the utilization of the transcytosis and uptake potential of the low-density lipoprotein (LDL) receptor as a means to deliver secretory proteins across the blood-brain barrier and to the lysosomes of neurons and astrocytes in the CNS.

In order to implement a gene delivery therapy for the treatment of Gaucher' s disease, a fusion construct was designed such that the glucocerebrosidase gene was fused at the N-terminus with the LDL receptor-binding domain of ApoB or ApoE.

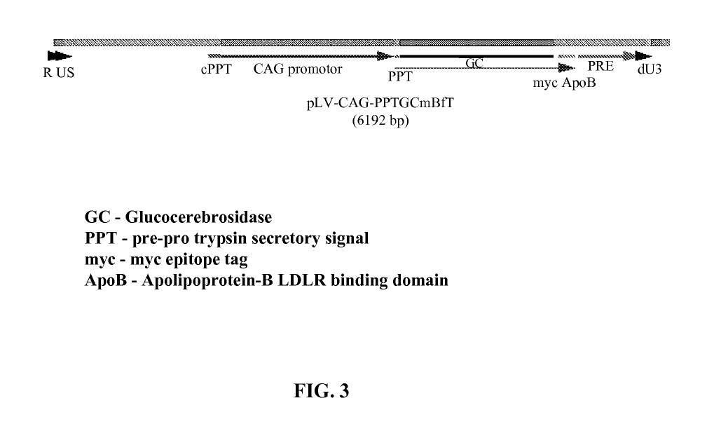

Briefly, the LDL receptor binding domain of Apolipoprotein B (ApoB) and Apolipoprotein E (ApoE) were fused to the C-terminus of the β-glucocerebrosidase (GC) gene along with the c-myc epitope tag. This construct was fused at the N-terminus to the secretory signal of pre-pro trypsin (PPT) to create the genes we labeled PPTGCmBfT or PPTGCmEfT. As a label for the two gene products together, the description herein is simplified by reference to the general term GCmXfT. A schematic diagram of the PPTGCmBfT is shown is FIG. 3.

GCmXft genes encoding a chimeric CNS targeting polypeptide were tested in a transfection protocol in vitro in which human embryonic kidney cells (293T) were transfected with the gene driven by the human cytomegalovirus (hCMV) promoter. Briefly, twenty-four hours after transfection, cells were washed twice with phosphate buffered saline (PBS) and plated onto screen-lined cups with a pore size of 0.4 μm. These cells were cultured in 6 well dishes coated on the bottom with human hepatocyte cells (HepG2) that had been grown in lipoprotein deficient serum in order to up-regulate the expression of the LDL receptor. Eighteen hours after co-culture of the two cell lines, the 293T cells plated in the cup were removed and the HepG2 cells were washed with PBS. These cells were then lysed and total cellular protein was separated on a 7% Tris-Acetate gel and probed with the anti-myc antibody to detect the GcmXfT gene.

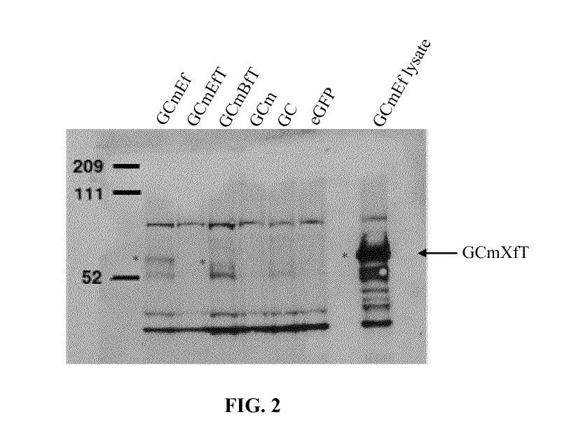

The results of the above-described co-culture of GCmXfT transfected cells expressing a ApoB or ApoE containing chimeric CNS targeting polypeptide with LDL receptor positive cells is shown in FIG. 2. This figure shows polypeptide levels expressed from the listed constructs that were bound and internalized by the LDL receptor into lysosomes. The polypeptide staining is of HepG2 lysates co-cultured with each of the respective 293 transfected cells. The results of FIG. 2 indicate that HepG2 cells co-cultured with 293T cells transfected with various GC constructs were able to take up the recombinant protein only when ApoE or ApoB LDL receptor binding domains were fused to the GC protein.

These PPTGCmXfT constructs were then inserted into the 3rd generation lentivirus vector under the control of the CAG promoter.

Briefly, the lentivirus is an icosahedral enveloped virus having a diploid RNA genome that becomes integrated into the host chromosome as a proviral DNA for genome replication. The lentiviral genome contains gag, pol and env genes which encode the structural polypeptides of the virion (p17, p24, p7 and p6); the viral enzymes protease, reverse transcriptase and integrase, and the envelope glycoproteins (gp120 and gp41), respectively. The lentiviral genome also encodes two regulatory polypeptides (Tat and Rev) and four accessory polypeptides that play a role in virulence (Vif, Vpu, Vpr and Nef). Unlike other retroviruses, lentiviruses have the ability to efficiently infect and transduce non-proliferating cells, including for example, terminally differentiated cells. Lentiviruses also have the ability to efficiently infect and transduce proliferating cells. Despite the pathogenesis associated with lentiviruses, it is well known to those skilled in the art that the undesirable properties of lentiviruses can be recombinantly separated so that its beneficial characteristics can be harnessed as a delivery vehicle for therapeutic or diagnostic genes. Therefore, lentiviral-based vectors can be produced that are safe, replication-defective and self-inactivating while still maintaining the beneficial ability to transduce non-dividing cells and integrate into the host chromosome for stable expression. A description of the various different modalities of lentiviral vector and packaging systems for vector assembly and gene delivery can be found in, for example, in Naldini et al., Science 272:263-267 (1996); Naldini et al., Proc. Natl. Acad. Sci. USA 93:11382-11388 (1996); Zufferey et al., Nature Bio. 15:871-875 (1997); Dull et al., J. Virol. 72:463-8471 (1998); Miyoshi et al., J. Virol. 72:8150-8157 (1998), and Zufferey et al., J. Virol. 72:9873-9880 (1998).

To produce the lentiviral vectors expressing PPTGCmXfT chimeric CNS targeting polypeptides, the packaging construct used was a split packaging genome system essentially as described by Dull et al., supra. Briefly, a tat defective packaging construct pCMVR8.93 was first generated by swapping an EcoRI SacI fragment from plasmid R7/pneo(−), Feinberg et al., Proc. Natl. Acad. Sci. USA, 88:4045-49 (1991), with the corresponding fragment of pCMVR8.91, a previously described plasmid expressing Gag, Pol, Tat, and Rev, Zufferey et al., Nat. Biotechnol., 15:871-75 (1997). This fragment has a deletion affecting the initiation codon of the tat gene and a frameshift created by the insertion of an MluI linker into the Bsu36I.

Next, pMDLg/p was generated, which is a CMV driven packaging construct that contains only the gag and pol coding sequences from HIV 1. First, pkat2Lg/p was constructed by ligating a 4.2 kb ClaI EcoRI fragment from pCMVR8.74 with a 3.3 kb EcoRI HindIII fragment from pkat2, Finer et al., Blood, 83:43-50 (1994), and a 0.9 kb HindIII NcoI fragment from pkat2 along with an NcoI ClaI linker consisting of synthetic oligonucleotides 5′ CATGGGTGCGAGAGCGTCAGTATTAAGCGGGGGAGAATTAGAT 3′ (SEQ ID NO: 4) and 5′ CGATCTAATTCTCCCCCGCTTAATACTGACGCTCTCGCACC 3′ (SEQ ID NO: 5). pCMVR8.74 is a derivative of pCMVR8.91, described above, in which a 133 bp SacII fragment, containing a splice donor site, has been deleted from the CMV derived region upstream of the HIV sequences to optimize expression. Second, pMDLg/p was constructed by inserting the 4.25 kb EcoRI fragment from pkat2Lg/p into the EcoRI site of pMD 2. pMD 2 is a derivative of pMD.G, Ory et al., Proc. Natl. Acad. Sci. USA, 93:11400-406 (1996), in which the pXF3 plasmid backbone of pMD.G has been replaced with a minimal pUC plasmid backbone and the 1.6 kb VSV G encoding EcoRI fragment has been removed.

Finally, packaging construct pMDLg/pRRE was produced, which differs from pMDLg/p by the addition of a 374 bp RRE containing sequence from HIV 1 (HXB2) immediately downstream of the pol coding sequences. To generate pMDLg/pRRE, the 374 bp NotI HindIII RRE containing fragment from pHR3 was ligated into the 9.3 kb NotI BgIII fragment of pVL1393 (Invitrogen, San Diego, Calif.) along with a HindIII BglII oligonucleotide linker consisting of synthetic oligonucleotides 5′ AGCTTCCGCGGA 3′ (SEQ ID NO: 6) and 5′ GATCTCCGCGGA 3′ (SEQ ID NO: 7) to generate pVL1393RRE (pHR3 was derived from pHR2 by the removal of HIV env coding sequences upstream of the RRE sequences in pHR2, where pHR2 is a transducing vector described below in Example II). A NotI site remains at the junction between the gag and RRE sequences. pMDLg/pRRE was then constructed by ligating the 380 bp EcoRI SstII fragment from pV1393RRE with the 3.15 kb SstII NdeI fragment from pMD 2FIX (pMD 2FIX is a human factor IX containing variant of pMD 2 which has an SstII site at the 3′ end of the factor IX insert), the 2.25 kb NdeI AvrII fragment from pMDLg/p, and the 3.09 kb AvrII EcoRI fragment from pkat1Lg/p, Finer et al., supra.

The second plasmid construct of the split packaging system consists of a nucleic acid vector expressing the rev gene product. pRSV Rev and pTK Rev are two such rev cDNA expressing plasmids in which the joined second and third exons of HIV 1 rev are under the transcriptional control of the RSV U3 and herpes simplex virus type 1 thymidine kinase (TK) promoters, respectively. Both expression plasmids utilize polyadenylation signal sequences from the HIV LTR in a pUC118 plasmid backbone. Dull et al., supra.

Lentiviral vectors packaging the PPTGCmXfT chimeric CNS targeting polypeptides were produced by co-transfection of the corresponding nucleic acid vectors together with a packaging construct. Transient transfection of the plasmid constructs into 293T cells was performed essentially as described by Naldini et al., Science 272:263-267 (1996). Briefly, a total of 5×10⁶ 293T cells were seeded in 10 cm diameter dishes 24 hours (h) prior to transfection in Iscove modified Dulbecco culture medium (JRH Biosciences) with 10% fetal bovine serum, penicillin (100 IU/ml), and streptomycin (100 μg/ml) in a 5% CO2 incubator, and the culture medium was changed 2 h prior to transfection. A total of 20 μg of plasmid DNA was used for the transfection of one dish: 3.5 μg of the targeting polypeptide plasmid hTf-CD40 or ApoE4-CD40 6.5 μg of packaging plasmid, and 10 μg of transducing vector plasmid.

The precipitate for transfection was formed by adding the plasmids to a final volume of 450 μl of 0.1×TE (1×TE is 10 mM Tris (pH 8.0) plus 1 mM EDTA) and 50 μl of 2.5 M CaCl2, mixing well, then adding dropwise 500 μl of 2× HEPES buffered saline (281 mM NaCl, 100 mM HEPES, 1.5 mM Na2HPO4 (pH 7.12)) while vortexing and immediately adding the precipitate to the cultures. The medium (10 ml) was replaced after 14 to 16 h; the conditioned medium was collected after another 24 h, cleared by low speed centrifugation, and filtered through 0.22 μm pore size cellulose acetate filters. For in vitro experiments, serial dilutions of freshly harvested conditioned medium were used to infect 105 cells in a six well plate in the presence of Polybrene (8 μg/ml). Viral p24 antigen concentration was determined by immunocapture using commercially available kits (Alliance; DuPont NEN). Vector batches were tested for the absence of replication competent virus by monitoring p24 antigen expression in the culture medium of transduced SupT1 lymphocytes for 3 weeks. In all cases tested, p24 was undetectable (detection limit, 3 pg/ml) once the input antigen had been eliminated from the culture. Transducing activity was expressed in transducing units (TU). Concentrated and purified lentiviral vector particles expressing the hTF-CD40 attachment polypeptide tested positive for the transferrin protein by protein blot.

Lentiviral vectors have been generated utilizing pseudotype polypeptides that exhibit a variety different cell type specificities. The pseudotype polypeptides utilized include, VSV-G, Rabies-G, HIV gp160, HIV gp⁴¹ and a binding deficient influenza hemagglutinin. The VSV-G fusion protein still retains the ubiquitous binding activity. The cell type specificity of VSV-G as well as the others described above are well known to those skilled in the art. The nucleic acid vector used for this transfection was pMD.G, Ory et al., supra. Incorporation was verified by harvesting lentiviral vector containing supernatant and concentrating by high speed centrifugation. The vector particles were further purified by centrifugation over a 20% sucrose cushion. The resulting lentiviral vector pellet was loaded onto a poly-acrylamide gel, electrophoresed and blotted to PVDF membrane.

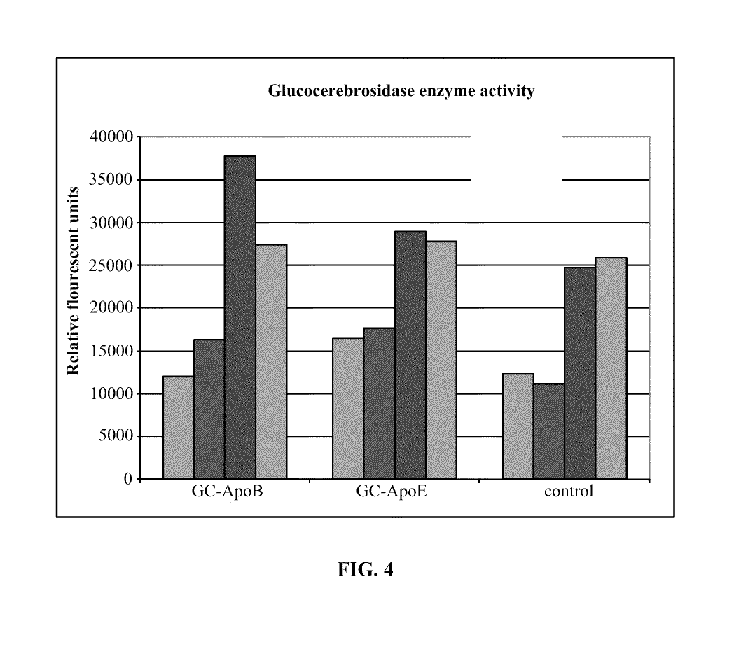

The viral particle harboring the PPTGCmXfT encoding constructs were generated via pseudotyping as described above. FIG. 4 shows the results utilizing viral vector particles with the VSV-G envelope following purification by centrifugation through a 20% sucrose cushion. Briefly, approximately 7×10⁸ tdu of each viral vector as determined by p24 ELISA assay, were injected via tail vein injection (i.e. intra-venously) into 4-6 week old BalB/C mice obtained from Jackson Laboratories. Seven and 14 days after virus delivery, serum samples were taken by retro-orbital bleeding. At 14 days after virus delivery, mice were sacrificed and liver and brain tissues were taken for analysis. Portions of the liver and brain were homogenized in cell lysis buffer and were examined for glucocerebrosidase enzyme activity as previously described. The results were analyzed on a fluorimeter and are shown in FIG. 4 as relative fluorescence units.

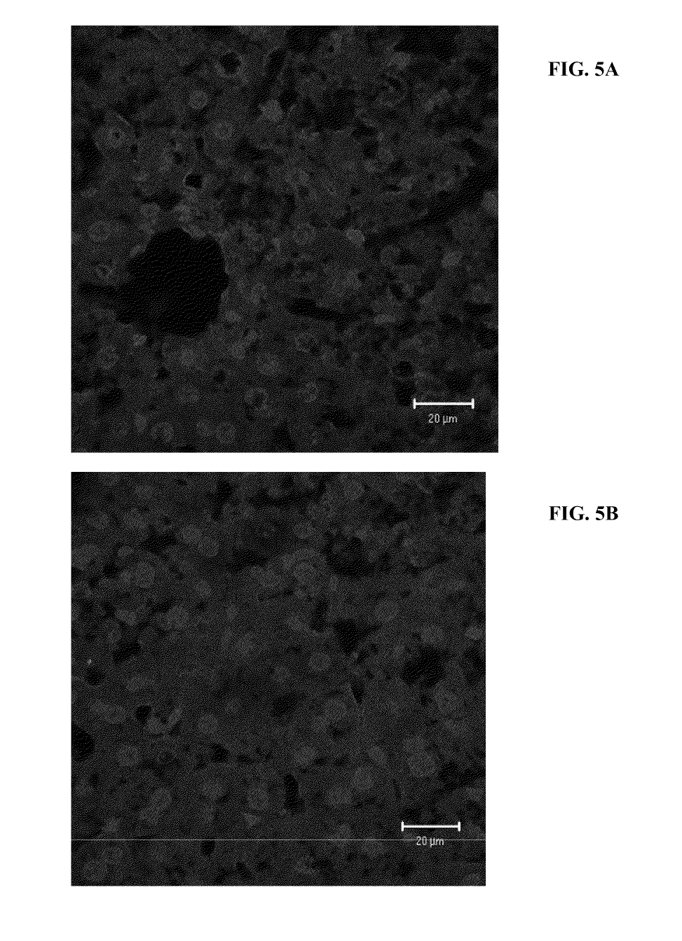

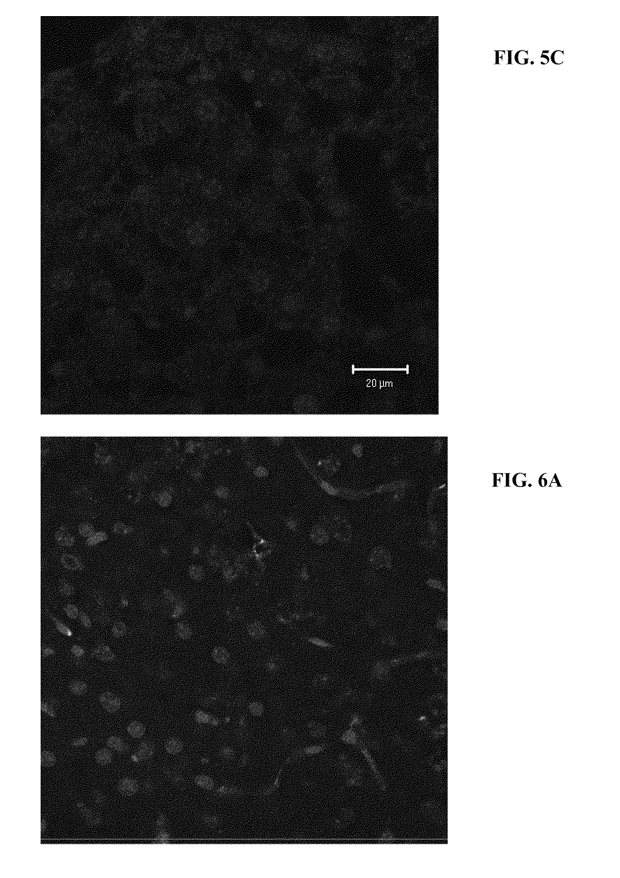

Portions of the liver and whole brain from the above intravenously injected animals were fixed in 4% paraformaldehyde for 2 hours at room temperature and then placed in 20% sucrose in PBS for 24 hours at 4 C. Liver tissues were mounted in OCT, frozen at B80 C and sectioned on a cryostat at 20 μm. The results are shown in FIG. 5. Briefly, sections from mice injected with the LV-GCmBfT (A) or LV-GCmEfT (B) or control (C) were stained with a mouse mono-clonal antibody for the myc tag of the GCmBfT or GCmEfT protein and were counterstained with TOPO-3 (blue) which stains the nuclei. Protein staining (red) was observed primarily in sinusoidal cells of the liver that are made up of endothelial cells, Kupffer cells, and ovoid cells.

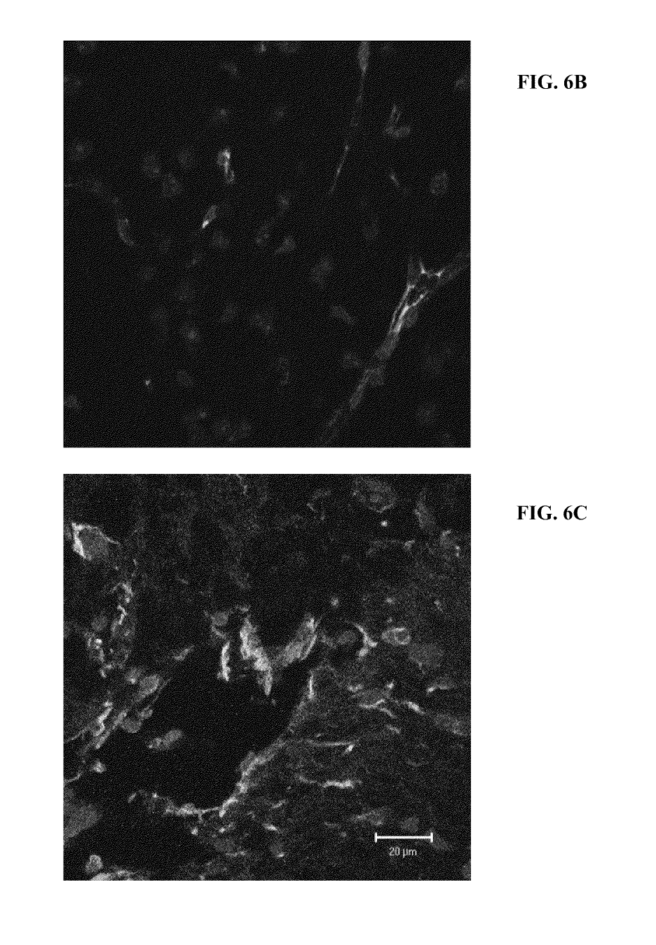

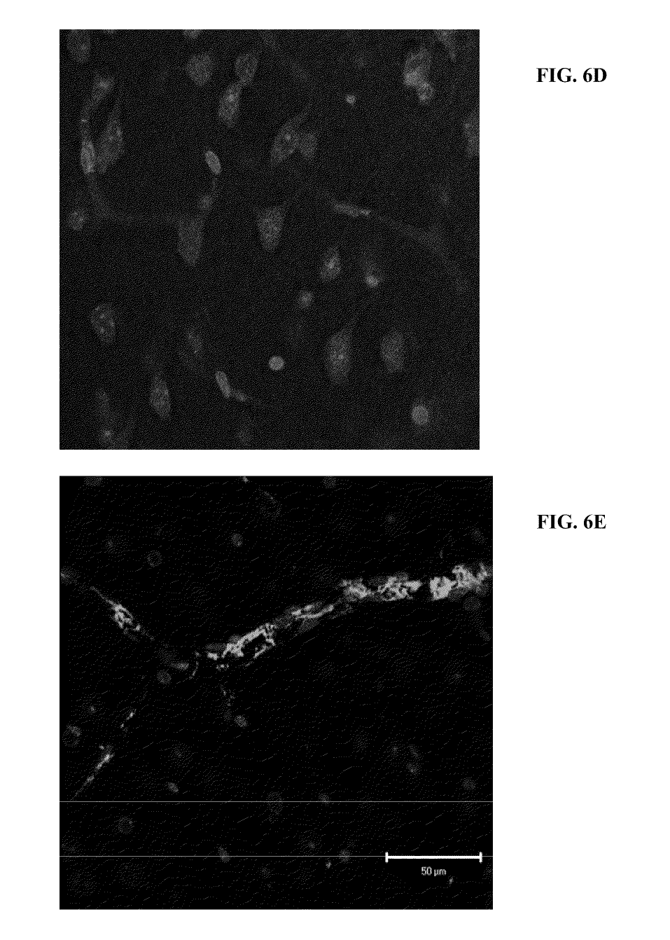

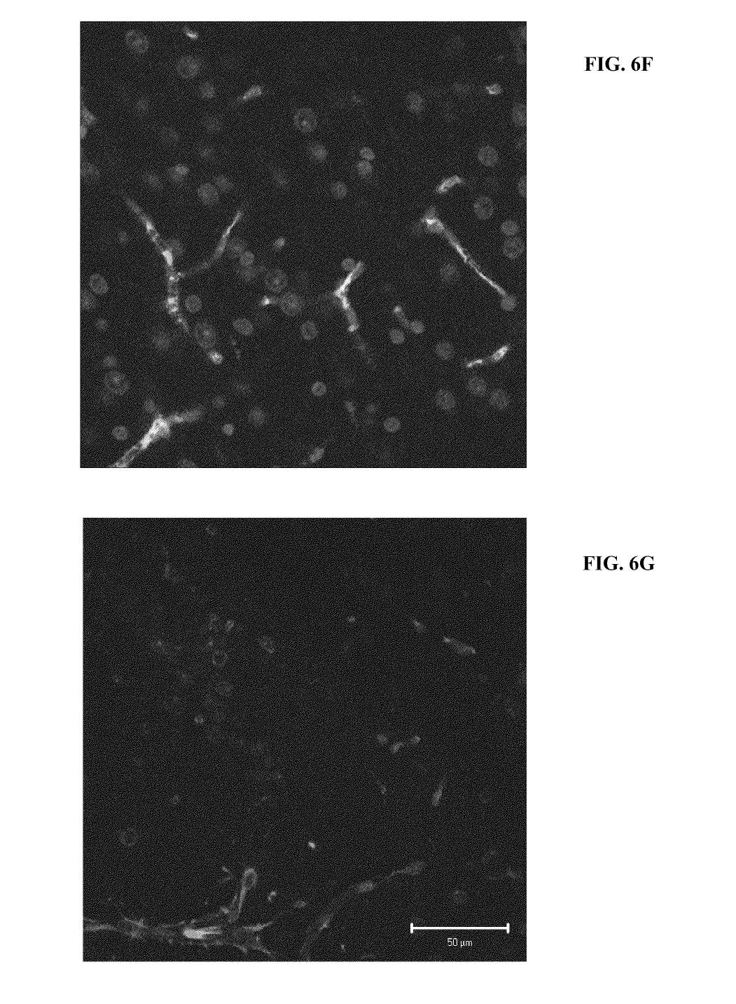

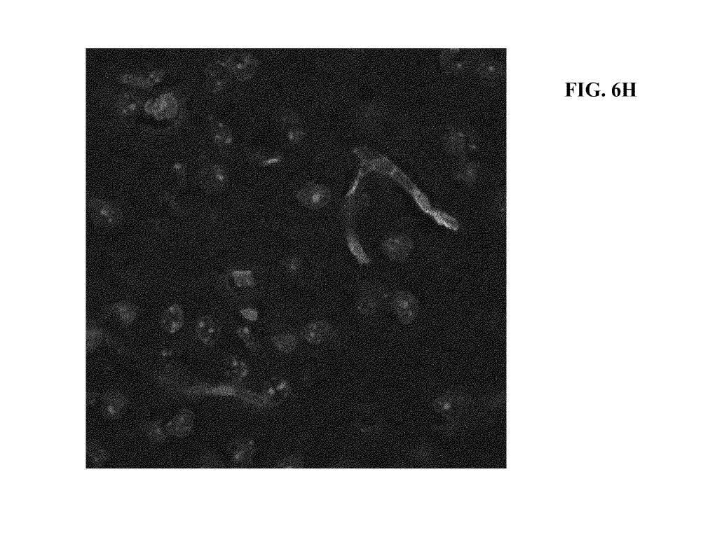

Brain tissues from the above intravenously injected mice were frozen with dry ice and sliced on a microtome at 50 μm thickness. Shown in FIG. 6 are sections stained for the myc tag (green) of the GCmBfT (A,B,C,D) or GCmEfT protein (E,F,G,H) and counterstained for various cellular markers (red): von-Willebrand factor (A,E), TuJ1 (B,F), GFAP (C,G) or LAMP1 (D,H) which label endothelial cells, neurons, astrocytes and lysosome organelles respectively. The TOPO-3 nuclear marker (blue) was used as a counterstain.

The results described above demonstrate delivery of the lentivirus vector expressing the GCmXfT gene via intra-venous route was successful at delivering the transgene to the sinusoidal cells of the liver thus making this organ a “depot organ” able to express and secrete the enzyme. The addition of the Apolipoprotein B or Apolipoprotein E LDL receptor binding domain was able to confer transport of the GC enzyme across the blood-brain barrier where it was it was taken up by neurons and astrocytes and correctly localized to the lysosomes.

In summary, the above-described constructs allowed targeting of the encoded protein for uptake via binding of the LDL receptor and transport to the lysosome. These constructs showed their ability in vitro to express and secrete enzymatically active glucocerebrosidase enzyme. In addition, cultured supernatant from transfected cells was applied to human hepatocytes, HepG2, expressing the LDL receptor. Western blot analysis of lysates from these HepG2 cells showed uptake of the enzyme. These constructs were then inserted into a 3rd generation lentivirus vector under the control of the murine CMV promoter (mCMV) or the CAG promoter. These viral vectors were delivered intra-venously into 4-6 week old BALB/c mice and 7 or 14 days later, blood and tissue samples were collected and analyzed. Serum from animals injected with the CAG glucocerebrosidase viruses showed increased enzyme activity compared to uninjected controls. Livers of all mice injected with either the mCMV or CAG glucocerebrosidase constructs contained recombinant enzyme as determined by Western blot. In addition, recombinant glucocerebrosidase could be detected in whole brain that was homogenized and subjected to Western blot analysis. Since previous reports have shown the lentivirus does not efficiently cross the blood-brain barrier, and an internal GFP expression construct in the virus was detected in the liver but not the brain, the results obtained demonstrate that the liver was functioning as a depot organ for expression of the glucocerebrosidase enzyme, which is then able to cross the blood-brain barrier following binding to the LDL receptor and translocation. These results further indicate that treatment can be effected for the neurological symptoms of Gaucher' s disease.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

1.-20. (canceled)

21. A composition comprising:
a first moiety comprising a receptor binding domain (RBD) which binds to a receptor on a cell of the blood brain barrier (BBB); and
a second moiety comprising a payload to be delivered across the BBB.

22. The composition of claim 21, wherein the RBD is a domain of an apolipoprotein.

23. The composition of claim 21, wherein the RBD is a domain of low density lipoprotein (LDL), transferin receptor, insulin receptor or insulin growth factor receptor.

24. The composition of claim 22, wherein the apolipoprotein is ApoB or ApoE.

25. The composition of claim 21, wherein the second moiety to be delivered is a therapeutic molecule.

26. The composition of claim 21, wherein the second moiety to be delivered is a toxin.

27. The composition of claim 21, wherein the composition is an enzyme.

28. The composition of claim 27, wherein the enzyme replaces or supplements a deficient lysosomal activity.

29. The composition of claim 21, wherein the composition is a fusion protein or a chemical conjugate.

30. The composition of claim 21, further comprising a secretory polypeptide or a tag.

31. An isolated nucleic acid molecule encoding the composition of claim 21.

32. A vector comprising the nucleic acid molecule of claim 31.

33. The vector of claim 32, wherein the vector is a virus.

34. The vector of claim 33, wherein the vector is a lentivirus, an adenovirus, a vaccinia virus or an adeno-associated virus (AAV).

35. A method of delivering a polypeptide to the CNS of a subject, comprising:
administering to a subject in need thereof, the composition of claim 21, thereby delivering a polypeptide to the CNS.

36. The method of claim 35, wherein the delivery is by injection or infusion.

37. The method of claim 35, wherein the subject has a disease or disorder.

38. The method of claim 37, wherein the disease or disorder is a lysosomal storage disease, a neurological disease, or CNS-associated disorder.

39. The method of claim 37, wherein the disease is Gaucher's disease, Alzheimer's disease, Fabry disease, Schnidler disease, Tay-Sachs disease or Parkinson's disease.

40. The method of claim 35, wherein the method comprises:
administering to the subject a vector comprising a polynucleotide sequence that encodes a chimeric polypeptide wherein the polypeptide comprises:
a secretory signal;
an apolipoprotein B (ApoB) low density lipoprotein (LDL) receptor binding domain; and
the polypeptide to be delivered to the CNS;
wherein the polynucleotide sequence comprising a secretory signal encoding nucleic acid sequence is expressed in a non-CNS cell, thereby allowing secretion of the chimeric polypeptide and delivering the polypeptide to the CNS.
